(19) United States
(12) Patent Application Publication
(10) Pub. No.: US 2021/0267802 A1
(43) Pub. Date: Sep. 2, 2021

(54) Eyelash Extension Protector

(71) Applicant: Nyree Ball, Canyon Country, CA (US)

(72) Inventor: Nyree Ball, Canyon Country, CA (US)

(21) Appl. No.: 16/874,838

(22) Filed: May 15, 2020

Related U.S. Application Data
(60) Provisional application No. 62/982,770, filed on Feb. 28, 2020.

(51) Int. Cl. A61F 9/02; A45D 44/12

(52) U.S. Cl. CPC A61F 9/027; A45D 44/12

(57) ABSTRACT

The present invention relates to an eyelash extension protector to protect eyelash extensions and false lashes from water, moisture and steam. The eyelash extension protector includes an eyewear frame and an adjustable strap. The eyewear frame comprises a pair of eye socket protectors. The eyewear frame is dimensioned to accommodate eyelashes and eyelash extensions, wherein the eyewear socket protector is wider and deeper where it covers the eye to accommodate longer and fuller eyelashes. The adjustable strap is configured to secure the eyewear frame in place.

### CROSS-REFERENCE TO RELATED APPLICATION

The present application claims priority to, and the benefit of, U.S. Provisional Application No. 62/982,770, which was filed on Feb. 28, 2020 and is incorporated herein by reference in its entirety.

### BACKGROUND

The present invention relates generally to eye covers or eyewear. More specifically, the present invention relates to an eyelash extension protector. Accordingly, the present specification makes specific reference thereto. However, it is to be appreciated that aspects of the present invention are also equally amenable to other like applications, devices, and methods of manufacture.

Typically, eyelash extensions or false eyelashes are applied to an eyelid to improve the appearance of a user by making it appear that the user has eyelashes of greater curl, color, fullness, length, etc., and eyelash extension users are cautioned to avoid showering within 24 hours after application or the steam could affect the adhesive used. It takes a lot of time for a user to keep up with a daily cosmetic routine of curling one's natural lashes and/or applying mascara, and further removing of mascara using mascara cleansers. Stated differently, the complete process of enhancing a user's eyelashes is both tedious and time consuming. Therefore, eyelash extensions have become increasingly popular in recent years. The eyelash extensions are applied to the eye-lids using any an eyelash extension adhesive that cures over time. Usually, the eyelash extensions last between two to three weeks after application. However, water, moisture and steam weaken the adhesive of the eyelash extension, and the extensions may fall out prematurely. At which time the user becomes frustrated that she paid for the eyelash extensions but did not get the full benefit thereof. Accordingly, most users tend to avoid washing their eyelash extensions within a reasonable amount of time as they want to protect their eyelash extensions from falling out and prolong the longevity thereof. Further, after the application of eyelash extensions, the user has to be always conscious about the after-care of the eyelash extensions depending on the type of glue or adhesive used.

Therefore, there exists a long-felt need in the art for an eyelash extension protector to protect the eyelash extensions and false eyelashes from various external factors such as water, steam, moisture, heat, etc. More specifically, there is a long-felt need in the art for an eyelash extension protector that allows individuals with eyelash extensions or any type of false eyelashes to get their face wet without the worry of the glue or adhesive coming undone. Further, there is a long felt need in the art for an eye protection device which prolongs the longevity of the eyelash extensions. There is also a long felt need in the art to eliminate or reduce the time consuming process of enhancing the eyelashes, and to save a considerable amount of time, effort and frustration in applying and removing mascara thereto. Finally, there is also a long felt need in the art to allow users to enjoy time spent in a hot tub, shower, swimming, or other steam filled area without ruining their extended eyelashes.

The subject matter disclosed and claimed herein, in one embodiment thereof, comprises an eyelash extension protector device for protecting the eyelash extensions and false eyelashes of a user from various external factors, such as water, steam, moisture, heat, and the like, and therefore increasing the useful life of the eyelash extensions. More specifically, the eyelash extension protector provides adjustable eye and eyelash protection to the user. Importantly, the present invention is adjustable, and allows a user to quickly and removably attach the eye protection to the user's head. The eye protection covers the eyelash and eyebrow area of the user, and conforms to a typical eye socket comfortably. Therefore, the eyelash extension protector prevents the eyelashes from falling out or coming undone prematurely, and extends the useful life thereof. In this manner, the eyelash extension protector of the present invention accomplishes all of the forgoing objectives, thereby improving the life of eyelash extensions and saving the user time, effort, and money in the process.

Further limitations and disadvantages of conventional and traditional approaches will become apparent to one of skill in the art through comparison of described systems with some aspects of the present disclosure, as set forth in the remainder of the present application and with reference to the drawings.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises an eyelash extension protector to guard eyelash extensions. More specifically, the eyelash extension protector is preferably comprised of an eyewear frame and an adjustable strap. The eyewear accommodates a user's eyelashes, wherein the eyewear frame is wider and deeper in the eye portion to accommodate longer and fuller eyelashes. The adjustable strap is configured to secure the eyewear frame in place. In one embodiment, the eyewear is designed to be worn by a user in a shower, a hot tub, steam room, sauna, swimming pool, beach, bathrooms, and during other water or steam related activities. In one embodiment, the eyewear is made of at least one of silicone rubber, natural rubber or similar materials. Additionally, an eye portion of the eyewear is transparent or translucent to facilitate the user to see through the device so as to not impair the user's vision.

In one exemplary embodiment, the eyewear frame comprises a pair of eye socket protectors and a bridge. The bridge connects the pair of eye socket protectors and engages the user's nose. The eyewear frame further comprises an inside periphery for conforming to a face of a user, and each of the pair of eye sockets comprises a sidewall, a receiving hole, and a lens. Furthermore, the sidewall comprises an upper portion and a lower portion, and is dimensioned to encapsulate eyelash extensions when in position on the user's face. Each receiving hole is positioned laterally in the sidewall.

Each lens may be convex or flat in shape and comprises an inside surface and an outside surface. Each lens may further comprise a protective layer on either the inside surface, the outside surface, or both. The protective layer is designed to protect each lens from fogging up, scratches, ultra-violet light, etc.

The strap comprises a pair of securing elements. The pair of securing elements attach the strap to the eyewear frame via the receiving holes in each pair of eye sockets. Each securing element may engage the respective receiving hole to form a watertight connection between the device and the user's face. The strap is adjustable, and may be elastic in nature. The strap may alternatively comprise an adjusting element for adjusting a length of the strap to fit a particular shape or size of a user.

To use the eyelash extension protector, the user would position the pair of eye socket protectors over the user's eyes without contacting the eyelash extensions. The user would then either pull the strap tight, relax the strap to engage the user's head, or adjust the adjusting element on the strap to ensure a tight fit between the eyelash extension protector and the user's face. The eyelash extension protector may further comprise a cushioning element. The cushioning element may be attached to the inside periphery of the protector via an adhesive, and would allow a more comfortable and better fit to a variety of individual user faces.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of the eyelash extension protector of the present invention and in accordance with the disclosed architecture.

FIG. 2 illustrates a perspective view of one potential embodiment of an eyewear portion of the eyelash extension protector of the present invention and in accordance with the disclosed architecture.

FIG. 3 illustrates a top perspective view of one potential embodiment of the eyelash extension protector of the present invention and in accordance with the disclosed architecture.

FIG. 4 illustrates an exploded side view of one potential embodiment of the eyewear portion of the eyelash extension protector of the present invention and in accordance with the disclosed architecture.

FIG. 5 illustrates a perspective view of one potential embodiment of the eyelash extension protector of the present invention being worn in a shower in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there exists in the art a long-felt need for an eyelash extension protector and a method for protecting eyelash extensions and false eyelashes from various external factors such as water, steam, moisture, heat, etc., and therefore protecting the extensions from falling out prematurely. There also exists in the art a long-felt need for an eyelash extension protector which increases the useful life of eyelash extensions, and keeps the eyelash extensions in place longer. The innovative product of the present invention features an eyelash extension protector which covers and protects eyebrows, eyelashes and eyelash extensions, in order to prevent them from getting exposed to water, steam, moisture, etc., and, therefore, permitting the eyelash extensions to remain intact on the user's eyelashes for a longer period of time.

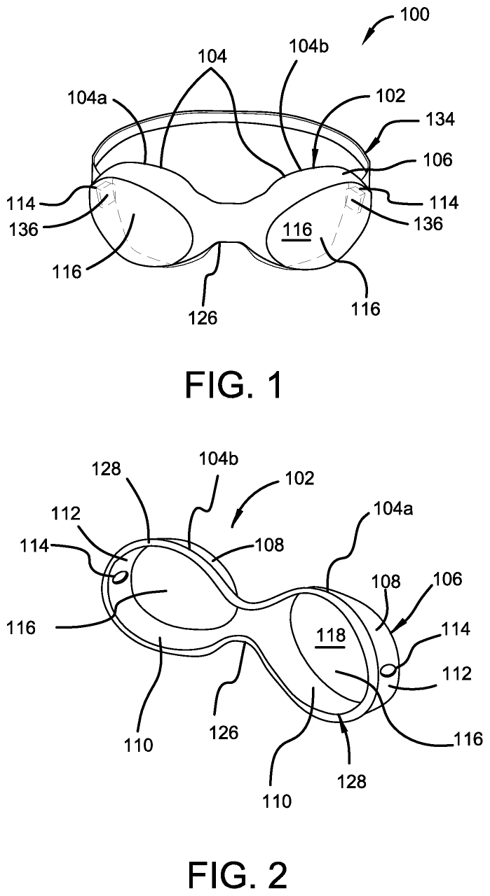

Referring initially to the drawings, FIG. 1 illustrates a front perspective view of the eyelash extension protector 100 in accordance with at least one embodiment. The eyelash extension protector 100 comprises an eyewear frame 102 and a strap 134. The eyewear frame 102 covers the eye sockets of a user like a pair of goggles, and accommodates eyelash extensions (i.e., does not interfere with the same). More specifically, the eyewear frame 102 comprises a pair of eye socket protectors 104, namely a right eye socket protector 104a and a left eye socket protector 104b. The right eye socket protector 104a and the left eye socket protector 104b are of sufficient width and depth in the eye portion to accommodate (i.e., not interfere with) longer and fuller eyelashes. Each of the eye socket protectors 104 may be oval, rounded, geometric, non-geometric, cylindrical, domed, or spherical in shape.

As best shown in FIGS. 1 and 2, the eyewear frame 102 further comprises a bridge 126, and an inside periphery 128. The bridge 126 connects the right and left eye socket protectors 104a and 104b in spaced apart fashion, and the inside periphery 128 is flexible to conform to the user's face. The eyewear frame 102 may be manufactured of a moldable transparent or generally translucent material such as silicone, polyethylene, polypropylene, polycarbonate, other types of moldable plastics, acrylic materials, fiberglass, glass, clear aluminum, resin, etc. The material used to form pair of eye socket protectors 104, is preferably relatively thin, non-collapsible, and able to maintain its form in warm, humid or moist conditions. The strap 134 is adjustable and is configured to secure the eyewear frame 102 in place on a user. In one embodiment, the adjustable strap 134 is a head strap that encircles a user's head.

As illustrated in FIG. 2, each eye socket protector 104a and 104b comprises a sidewall 106, a receiving hole 114, and a lens 116. Each sidewall 106 further comprises an upper portion 108, a lower portion 110, and a lateral portion 112. The sidewall 106 is typically at least 10-15 mm deep to accommodate most eyelash extensions that may be attached to a user's eyelashes. Each receiving hole 114 is located in the lateral portion 112 of the respective eye socket protector 104a and b, the purpose of which is described more fully below.

As previously stated, the strap 134 is adjustable, and is releasably securable to the eyewear frame 102 through the respective receiving holes 114 therethrough. The strap 134 comprises a plurality of securing elements 136 at each end for engaging said receiving holes 114. The securing elements 136 utilize a securing mechanism, such as a threading knot, to attach the adjustable strap 134 to the eyewear frame 102. In an alternate embodiment, other securing mechanisms such as a threading nut or other fastening mechanisms can be used.

Furthermore, it may be desirable for the material of the eyewear frame 104 to be generally lightweight, so that very little pressure is felt around the eye area of the user, which could result in skin irritation or deterioration for the user, and even the development of pressure sores over time. Many embodiments of the present invention are not limited to a particular shape, size, depth, density, composition, and durability of the eyewear. Those skilled in the art will readily recognize, in light of and in accordance with the teachings of the present invention, that in some embodiments alternate methods may be used to form the eyewear structure including, without limitation, vacuum forming, thermoforming, machining, etc., and in such embodiments, non-moldable materials may also be used. The materials disclosed are designed to prevent water, moisture and/or steam from entering the interior space of the eyewear 102 (i.e., the space between the eyewear frame 104 and the user's face). Depending on the material and manufacturing method chosen to make the eyewear, some embodiments may be intended for one-time usage and then disposal while others may be intended to offer multiple usages.

The bridge 126 is capable of resting on the user's nose to permit the eyelash extension protector 100 to be worn by the user. Accordingly, the eyewear frame 102 is configured to fully cover the false eyelashes or eyelash extensions of the user. The right eye socket protector 104a and the left eye socket protector 104b shield the false eyelashes against water, moisture, shampoo, dust, and the like. After each use, the eyelash extension protector 100 may be hung for drying or otherwise stored until needed again.

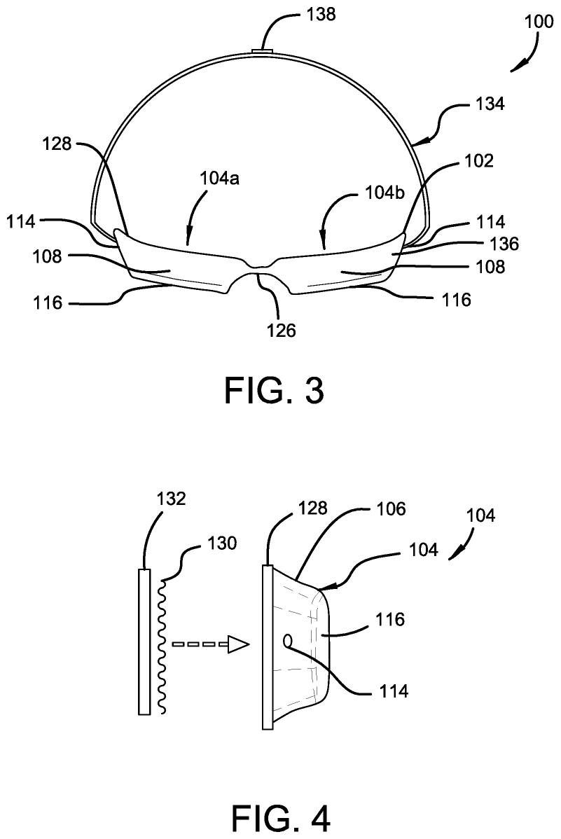

As further illustrated in FIGS. 2 and 3, the eye socket protectors 104a and 104b are typically transparent to facilitate the user to visualize the surroundings. The eye socket protectors 104a and 104b include the receiving holes 114 respectively, which utilizes a securing mechanism such as a threading knot to attach the adjustable strap 134. Each securing element 136 may engage the respective receiving hole 114 to form a generally watertight connection. Further, the upper portion 108 of the sidewall 106 covers the eyebrows of the user; and the lower portion 110 covers where the eyewear frame 102 will touch the user's face below the bottom eyelashes of the user's eyes. According to an embodiment herein, the transparent eye socket protectors 104a and 104b are fitted within the eyewear frame 102 and are significantly shaped in a bubble or convex configuration to ensure that the transparent eye socket protectors 102 do not interfere with the eyelash extensions of the user.

The depth of the sidewall 106 of the transparent eye socket protectors 104a and 104b may be deep enough to typically ensure that eyelash extensions do not touch the eye socket portions, whereby protecting extensions from being bent while still offering protection for eyelash extensions from outside interference. In addition, the eye socket protectors 104a and 104b typically have a relatively small footprint, covering only a small portion of the face of a user. The eye socket protectors 104a and 104b may be of any shape such as cylindrical, spherical, dome, or other, as suitable to user needs.

According to a preferred embodiment of the present invention, each lens 116 comprises an inside surface 118 and an outside surface 122. Each lens 116 may be convex or flat in configuration, and may comprise a protective layer that is comprised of one or more of the following coatings: an anti-fogging coating, a UV protection coating, and a water-shedding or repellant coating. In one preferred embodiment, the inside surface 118 has a fog-resistant coating. In another preferred embodiment, each lens 116 has UV protection incorporated therein, and the outside surface 122 may have a water-shedding coating. The material of each lens 116 may be coated to resist fogging as well as protect against UV penetration. According to an embodiment herein, each lens 116 may be treated with an anti-fogging agent in order to prevent the user's view from becoming obstructed. Additionally, each lens 116 may be scratch resistant.

Additionally, the pair of eye socket protectors 104 are preferably non-porous in nature and act as a barrier to heat, e.g., dry heat emitted from opening a hot oven, or heat from a steam room that may otherwise damage or melt the adhesive use to attach the false eyelashes to the user's eyelashes. An adhesive and/or cushioning may also have waterproofing attributes that may further contribute as a form of barrier not only from water and steam but also from the heat in similar circumstances. The material of the pair of eye socket protectors 104, pads and adhesive are not limited in its properties and may also include some form of heat reflective, or heat-insulating properties. Thus, it is contemplated that the eyelash extension protector 100 may also be used effectively in a steam room, sauna or hot bath. Furthermore, in some applications, users may use the eyelash extension protector 100 in other scenarios such as, but not limited to, bathroom, gym settings, swimming in a pool, on the beach, and during other similar water related activities. Moreover, some embodiments of the present invention may be implemented with numerous additional and alternative features such as, but not limited to, various designs on the pair of eye socket protectors 104 to add a whimsical or decorative touch, etc. The eyewear 102 may also include a sealant or a thin layer of cushioning pad placed around a periphery 124 of the right eye socket portion 102a and the left eye socket portion 102b to make the eyewear more comfortable to wear.

As noted above, the adjustable head strap 134 is adjustable in length and is designed to be placed below the hairline of the user. In one embodiment, the head strap 134 is a rubber strap that is long enough to extend right around the user's head. The head strap 134 may be elastomeric, elasticated cord or ordinary cord. The material of the head strap 104 allows for a snug fit, creating a shower proof seal. The strap 134 may further comprise an adjusting means 138. In an embodiment, the head strap 134 can be continuous or may include a slide adjustment. In an alternative embodiment, the adjustable strap 134 is composed of two Velcro® straps originating from the receiving holes 114 such that a fastener known in the art such as a Velcro® fastener may be used to fasten the two Velcro® straps of the head strap 134. The design of the head strap 134 allows for a snug fit along with and just below the entire hairline, thereby creating a shower proof seal. Further, to remove the eyewear frame 102, the Velcro® straps may be detached.

In an embodiment, the strap 134 may further comprise an adjusting means 138, such as a buckle, hook, clip or other tightening or fastening mechanism, or have free ends for tying. In one embodiment, the head strap 134 is a flexible strap made of a double latex strap. In another embodiment, the head strap 134 may have a plurality of colors such as grey, black, white, red or may include various color changing mechanisms. The color-changing mechanism may allow for different colors with different ray luminance to be visible in different settings (e.g., indoor or outdoor). In one embodiment, the head strap 134 further provides a desirable surface which may have corporate logos or other graphics and/or designs. For example, the corporate logos or other graphics may be positioned at the rear of the user's head where visibility to others will be maximized, or may be affixed to the sides of the head strap 134. Further, a name of the user may also be printed on the head strap 134 itself in order to personalize the eyelash extension protector 100 to suit user need and/or preference.

The inside periphery 128 may be strategically shaped to conform to a typical eye socket comfortably. In addition, the depth of the pair of eye socket protectors 104 may be sufficiently deep to reduce the possibility of eyelash extensions being bent while the device is worn. Further, the depth of the pair of eye socket protectors 104 may be designed to accommodate virtually any length of eyelash extensions. In some embodiments, eyelash extension protector 100 may be made available with eyewear socket portions 104 of different depths to enable a user to choose the depth that is appropriate for them.

As best illustrated in FIG. 4, the eyewear extension protector 100 may further comprise a cushioning element 132. The cushioning element 132 may be attached to the inside periphery 128 of the eyewear frame 102, and may include a sealant or a thin layer of cushioning pad placed around the periphery 128. The sealant or the cushioning pad 132 is adhered to the periphery 128 by utilizing an adhesive 132. The sealant may be a gel-based, fluid-tight sealant comfortable to a user's face while under tension, thereby creating a watertight seal and a controllable airflow seal. Similarly, thin layer of cushioning pad 132 may be comprised of a material which is comfortable to the user's face while under the tension, thereby creating a watertight seal and a controllable airflow seal. In one preferred embodiment, the eyewear frame 102 comprises a plurality of pads assembled on the inside periphery 128, wherein the plurality of pads is made of a soft material for providing comfort for the user. It is contemplated that various materials may be used for said padding or cushioning including, without limitation, foam, silicone, rubber, corrugated fiberboard pads, molded pulp, forms of inflatable air cushioning, etc. In a further preferred embodiment, the cushioning 132 may include a thin layer of waterproof and/or pressure-sensitive skin adhesive to connect the eye socket protectors 104 to the surface skin of the eye socket of the user and comfortably cover the eyelashes and eyelash extensions. More specifically, the cushioning and the adhesive may work together to offer a comfortable and secure seal between the eye socket portion and the user's skin to effectively protect the user's eyelash extensions from water, steam, moisture, heat, and the like. In an exemplary embodiment, the adhesive may be hypoallergenic and/or latex-free to reduce the possibility of an allergic response from the user, and of a grade that is suitable and conformable for the delicate eye area. Other alternate embodiments may be implemented without padding in which the skin adhesive is located directly on the inside periphery 128.

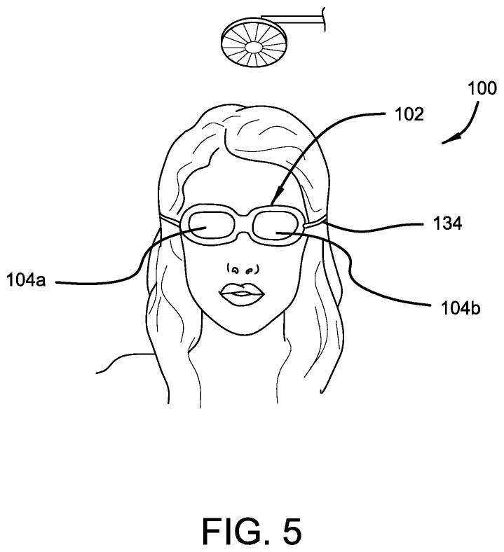

FIG. 5 illustrates a perspective view of one potential embodiment of the eyelash extension protector 100 being worn by a user during a shower and in accordance with the disclosed architecture. More specifically, FIG. 5 illustrates an exemplary eyelash extension protector 100 as applied to the eye areas of a user, wherein the right eye socket protector 104a covers the user's right eye and the left eye socket protector 104b covers the left eye of the user. The right eye socket protector 104a and the left eye socket protector 104b are comprised of a cushioning pad around the periphery of the eyewear, which may also have waterproofing attributes that may further contribute as a form of barrier not only from water and/or steam from the shower, but also from the accompanying heat. In an alternative embodiment, the cushioning may not be required and periphery 128 of the eyewear frame 102 may naturally stick to the face of the user and achieve the same objective.

In a preferred embodiment of the present invention, the upper portion 108 of the sidewall 106 will cover the eyelash extensions and the eyebrows of the user, and the lower portion 110 of the sidewall 106 of the eye wear 102 will contact or touch the user's face below the bottom eyelash extensions of the eye of the user. Accordingly, the device will cover both the top and bottom eyelash extensions of the user's eyes. In the present embodiment, the head strap 134 in combination with the eyewear frame 102 conforms comfortably to the orbital bone of the user, and may offer a secure bond between the eye socket cover and the skin of the user. Further, the relatively small footprint of eyewear frame 102 along with the head strap 134 that goes around the back of the user's head may allow the user to wash her hair and face comfortably and thoroughly. Moreover, the depth of the sidewall 106 is preferably deep enough to ensure that the glasses do not come into contact with the eyelash extensions, thereby both preserving and protecting the same from outside interference.

The eyelash extension protector 100 is designed to be worn by the use in the shower, hot tubs, swimming pool, bathrooms, etc., and protects various cosmetics such as eyelash extensions, false-eyelashes and/or mascara applied to the user's eyelashes from getting exposed to moisture, water, heat, dust and the like. Further, the eyelash extension protector 100 can be used by users while swimming, shampooing their and/or washing their face. In one preferred embodiment, the eyelash extension protector 100 may be designed to reduce the overall hydrodynamic drag when the user is swimming while wearing the protector 100.

Throughout this specification the terms “eyewear socket cover”, “eyewear socket portion” and “eye socket cover” may be used interchangeably to refer to protecting covers of eyewear. Also, the terms “adjustable strap”, “head strap” and “headband” are used interchangeably.

Notwithstanding the foregoing, the eyelash extension protector 100 of the present invention and its various structural components can be any suitable size, shape, and configuration as is known in the art without affecting the overall concept of the invention, provided that it accomplishes the above-stated objectives. One of ordinary skill in the art will appreciate that the shape and size of the eyelash extension protector 100 and its various components, as shown in the FIGS. are for illustrative purposes only, and that many other shapes and sizes of eyelash extension protector 100 are well within the scope of the present disclosure. Although dimensions of eyelash extension protector 100 and its components (i.e., length, width, and height) are important design parameters for good performance, the eyelash extension protector 100 and its components may be any shape or size that ensures optimal performance during use and/or that suits user needs and/or preference.

Those skilled in the art will recognize, in accordance with the teachings of the present eyelash extension protector and method, that some embodiments may be used in a wide variety of scenarios to protect the eye area, whether a person has eyelash extensions or not. For example, without limitation, some embodiments may be used to protect eyes and extensions during a facial, as oftentimes steam is involved. Some embodiments may also be used to protect the eyes following a cosmetic tattoo procedure, such as, but not limited to, the application of permanent eyeliner. Some embodiments may be used to protect one's eye makeup including, but not limited to, mascara, eyeliner and eyeshadow from water, steam, heat, and the like. In some embodiments, SPF (Sun Protection Factor) and UV (ultraviolet) protection may be added to the protective eye cover for use in tanning beds, on the beach, or poolside, which may cause the user to perspire.

In a further preferred embodiment, the eyelash extension protector of the present invention may utilize antiglare properties and polarization. Additionally, the present eyelash extension protector does not cause puffiness, bruising, laxity of the eye-lids, capillary breakage, dark circles, deep lines and loose skin relative to the user, and may also protect eyelash extensions from perspiration during various different types of activities such as, but not limited to, aerobic exercises, hiking, sports, yard work, etc.

The present invention provides protection to eyebrows along with eyebrow extensions. Similarly, to eyelash extensions, eyebrow extensions also require protection from water, steam, heat, etc., particularly within the first 48 hours after application. Eyebrow extension covers can also be used after a micro-blading procedure, which is a manual method used to deposit small amounts of ink in the superficial layer of the skin, similar to a semi-permanent tattoo or following a more permanent tattoo cosmetic procedure done on the brows.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An eyelash extension protector comprising:
an eyewear frame comprising:
a pair of eye socket protectors;
a bridge connecting the pair of eye socket protectors; and
an inside periphery; and
a strap comprising a pair of securing elements for attaching the strap to the eyewear frame.

2. The eyelash extension protector of claim 1, wherein each of the eye socket protectors are oval in shape.

3. The eyelash extension protector of claim 1, wherein the strap is adjustable in length.

4. The eyelash extension protector of claim 1, wherein each securing element is a threaded knot.

5. The eyelash extension protector of claim 1, wherein each eye socket protector comprises a sidewall, a receiving hole, and a lens.

6. The eyelash extension protector of claim 5, wherein each sidewall is at least 15 millimeters deep.

7. An eyelash extension protector comprising:
an eyewear frame comprising:
a pair of eye socket protectors, each eye socket protector comprising a sidewall, a receiving hole, and a lens;
a bridge connecting the pair of eye socket protectors; and
an inside periphery; and
a strap comprising a pair of securing elements for attaching the strap to both of the receiving holes.

8. The eyelash extension protector of claim 7, wherein each sidewall comprises an upper portion and a lower portion.

9. The eyelash extension protector of claim 7, wherein each securing element engages the respective receiving hole to form a watertight connection.

10. The eyelash extension protector of claim 7, wherein each lens is convex.

11. The eyelash extension protector of claim 7, wherein each lens is flat.

12. The eyelash extension protector of claim 7, wherein each lens is scratch resistant.

13. The eyelash extension protector of claim 7, wherein each lens comprises a protective layer.

14. The eyelash extension protector of claim 7, wherein the inside periphery is flexible.

15. The eyelash extension protector of claim 7, wherein the strap further comprises an adjusting element.

16. An eyelash extension protector comprising:
an eyewear frame comprising:
a pair of eye socket protectors, each eye socket protector comprising a sidewall, a receiving hole, and a lens;
a bridge connecting the pair of eye socket protectors; and
an inside periphery; and
a strap comprising a pair of securing elements for attaching the strap to both of the receiving holes; and
a cushioning element attached to the inside periphery of the eyewear frame.

17. The eyelash extension protector of claim 16, wherein the cushioning element is attached to the inside periphery of the eyewear frame by an adhesive.

18. The eyelash extension protector of claim 16, wherein each lens comprises a protective layer.

19. The eyelash extension protector of claim 16, wherein each sidewall is at least 15 millimeters deep.

20. The eyelash extension protector of claim 16, wherein the strap further comprises an adjusting element.
